(19) United States
(12) Patent Application Publication
(10) Pub. No.: US 2014/0154722 A1
(43) Pub. Date: Jun. 5, 2014

(54) APPARATUS FOR ANALYZING BIOMATERIAL

(71) Applicants: Moo-Yeal Yeal, Burlingame, CA (US); Suk Ho Song, Asan-si (KR); Dong Woo Lee, Suwon-si (KR); Se Hoon Jeong, Seongnam (KR); Bo Sung Ku, Suwon-si (KR)

(72) Inventors: Moo-Yeal Yeal, Burlingame, CA (US); Suk Ho Song, Asan-si (KR); Dong Woo Lee, Suwon-si (KR); Se Hoon Jeong, Seongnam (KR); Bo Sung Ku, Suwon-si (KR)

(73) Assignee: Solidus Biosciences, Inc.

(21) Appl. No.: 13/818,919

(22) PCT Filed: Aug. 10, 2012
(86) PCT No.: PCT/US12/50426

§ 371 Date: Jun. 6, 2013

Related U.S. Application Data
(60) Provisional application No. 61/522,750, filed on Aug. 12, 2011.

(51) Int. Cl. G01N 33/50; B01L 3/00

(52) U.S. Cl. CPC G01N 33/5008; B01L 3/5085
USPC 435/25; 435/289.1; 422/552; 435/174; 435/29

(57) ABSTRACT

There is provided an apparatus for analyzing a biomaterial. The apparatus includes: a first substrate including a plurality of micro-pillars formed to protrude to a predetermined height, the biomaterial being attached to one surface of the micro-pillar; a second substrate including a plurality of micro-wells, the micro-pillars being insertable into the micro-wells when the first substrate-and the second substrate are combined with each other; and at least one spacer disposed between the first substrate and the second substrate when the first substrate and the second substrate are combined with each other.

### BACKGROUND OF THE INVENTION

1. Field of the Invention

The present invention relates to an apparatus for analyzing biomaterial, and more particularly, to an apparatus for analyzing biomaterial capable of obtaining accurate test results.

2. Description of the Related Art

Recently, a demand for biomedical devices and biotechniques for rapidly diagnosing various human diseases is increasing. Since the results of existing tests for specific diseases performed in hospitals and laboratories may take a long time to be returned, the development of biosensors or biochips capable of providing test results in a timely manner is actively progressing.

Research into biosensors and biochips is required in hospitals as well as in pharmaceutical companies, cosmetics companies, or the like. A method of testing the response of cells to specific medications to verify the effectiveness and safety (toxicity) thereof is used in the pharmaceutical and cosmetics fields. Existing testing methods have shortcomings in that they are time-consuming and expensive, since animals and large amounts of reagent have to be used therein.

Accordingly, the development of biosensors and biochips for allowing for cost reductions and diagnoses which are simultaneously rapid and accurate are required.

Biochips can be sorted into DNA chips, protein chips, and cell chips, according to the kind of biomaterial fixed to a substrate. In early research, DNA chips were prominent, in concern with understanding about human genetic information. However, as interest in the proteins underlying life and the way in which cells become the backbone of a living thing as a corporate body of proteins is increasing, new interest has been shown in protein chips and cell chips.

The development of biochips capable of obtaining more accurate experimental results at lower cost, regardless of the specific kinds of biomaterials is therefore required. In order to achieve this, it is necessary to accurately and effectively perform the storage of the biomaterials attached to the biochips and the supplying of culture media and reagents.

### SUMMARY OF THE INVENTION

An aspect of the present invention provides an apparatus for analyzing a biomaterial capable of obtaining accurate test results.

According to an aspect of the present invention, there is provided an apparatus for analyzing a biomaterial, the apparatus including: a first substrate including a plurality of micro-pillars formed to protrude to a predetermined height, the biomaterial being attached to one surface of the micro-pillar; a second substrate including a plurality of micro-wells, the micro-pillars being insertable into the micro-wells when the first substrate and the second substrate are combined with each other; and at least one spacer disposed between the first substrate and the second substrate when the first substrate and the second substrate are combined with each other.

The spacer may be formed on the second substrate.

The spacer may be formed on the second substrate, the spacer being surface-contacted or point-contacted with the first substrate.

The first substrate may further include a penetration hole formed between the micro-pillars thereof.

The spacer may be formed at the edge portion of the first substrate or the second substrate.

The spacer may be formed on the second substrate, and the first substrate may further include a penetration hole formed between the micro-pillars thereof.

The spacer may be formed on the second substrate, and the first substrate may further include a guide groove into which a portion of the spacer is inserted, the guide groove being formed at a position of the first substrate corresponding to the spacer.

The guide groove may have a width varying in a depth direction thereof, the width of one region of the guide groove being equal to the width of the spacer such that only the portion of the spacer is inserted into the guide groove.

The spacer may have a width varying in a height direction, the width of one region of the spacer being equal to the width of the guide groove, such that only a portion of the spacer is inserted into the guide groove.

The guide groove may have a depth smaller than the height of the spacer.

The guide groove may be formed to penetrate the first substrate.

The width of the guide groove and the width of the spacer may vary in a height or depth direction thereof, the width of one region of the guide groove being equal to the width of one region of the spacer such that the portion of the at least one spacer is inserted into the guide groove.

### BRIEF DESCRIPTION OF THE DRAWINGS

The above and other aspects, features and other advantages of the present invention will be more clearly understood from the following detailed description taken in conjunction with the accompanying drawings, in which:

FIG. 1A and FIG. 1B are schematic perspective views showing an apparatus for anaylzing a biomaterial according to an exemplary embodiment of the present invention;

FIG. 2 is a perspective view showing an interaction between a first substrate and a second substrate in an apparatus for anaylzing a biomaterial according to an exemplary embodiment of the present invention;

FIG. 3 is a cross-sectional view showing an apparatus for analyzing a biomaterial according to another exemplary embodiment of the present invention;

FIG. 4 is a cross-sectional view showing a portion of an apparatus for analyzing a biomaterial according to another exemplary embodiment of the present invention;

FIG. 5A and FIG. 5B are plan views showing a first substrate and a second substrate according to an exemplary embodiment of the present invention;

FIG. 6 is a cross-sectional view showing a portion of an apparatus for anaylzing a biomaterial according to another exemplary embodiment of the present invention;

FIG. 7 is an enlarged cross-sectional view showing the structures of a spacer and a guide groove;

FIG. 8 is a cross-sectional view showing an apparatus for anaylzing a biomaterial without spacers and guide grooves different from the exemplary embodiment of the present invention; and

FIGS. 9A to 9C are cross-sectional views showing a spacer and a guide groove according to another exemplary embodiment of the present invention.

### DETAILED DESCRIPTION OF THE PREFERRED EMBODIMENT

Exemplary embodiments of the present invention will now be described in detail with reference to the accompanying drawings. Exemplary embodiments of the present invention may be modified in many different forms and the scope of the invention should not be seen as being limited to the embodiments set forth herein. Rather, these embodiments are provided so that this disclosure will be thorough and complete, and will fully convey the concept of the invention to those skilled in the art. In the drawings, the shapes and dimensions may be exaggerated for clarity, and the same reference numerals will be used throughout to designate the same or like components.

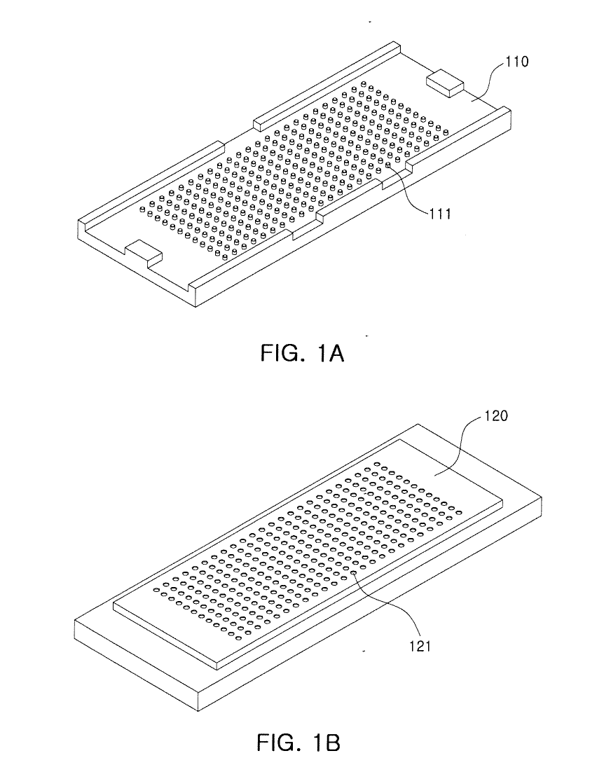

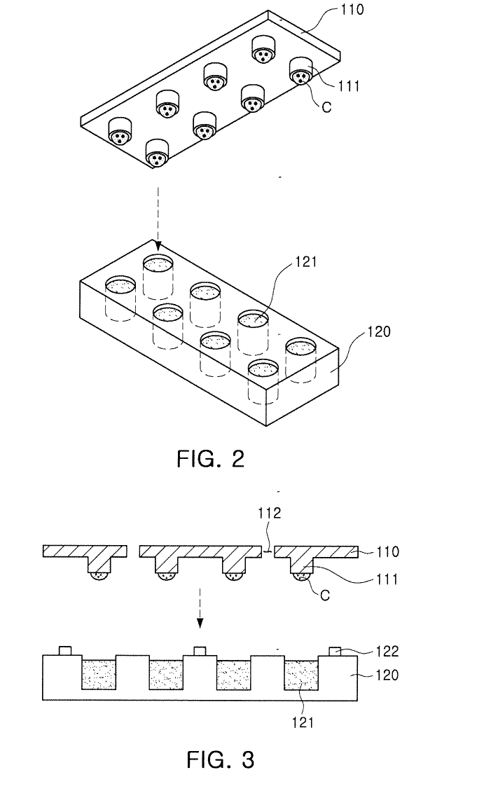

FIGS. 1A and 1B are schematic perspective views showing an apparatus for anaylzing a biomaterial according to an exemplary embodiment of the present invention. More specially, FIG. 1A is a perspective view showing a first substrate constituting the apparatus for anaylzing a biomaterial, and FIG. 1B is a perspective view showing a second substrate constituting the apparatus for anaylzing a biomaterial. FIG. 2 is a perspective view showing an interaction between the first substrate and the second substrate in the apparatus for anaylzing a biomaterial according to the exemplary embodiment of the present invention.

Referring to FIG. 1A, FIG. 1B, and FIG. 2, an apparatus for anaylzing a biomaterial according to an exemplary embodiment of the present invention may include a first substrate 110 and a second substrate 120. A plurality of micro-pillars 111 may be formed in the first substrate 110, and micro-wells 121 may be formed in the second substrate 120 at positions corresponding to the micro-pillars 111. Spacers may be disposed between the first substrate and the second substrate, and in the present exemplary embodiment, a plurality of spacers are formed in the second substrate 120 as an example.

The micro-pillar 111 may be a structure protruding to a predetermined height from one surface of the first substrate 110, which may be a fine rod and a fine pin. The micro-pillar 111 may be a three-dimensional structure, and a biomaterial may be attached to one surface of the micro-pillar 111.

The micro-pillar may have one of various heights. The micro-pillar may be, but not limited to, for example, 50 to 500 μm. Also, the cross-section and the surface shape of the protruding micro-pillars are not particularly limited. The micro-well 121 may be formed on one surface of the second substrate 120 to have a predetermined depth, and may be provided as a fine groove. The width and depth of the micro-well 121 may not be particularly limited, and may be selected from a proper range so that the micro-pillar can be inserted into the micro-well 121.

The first substrate and the second substrate used in the present invention are not particularly limited, and may be formed of, for example, silicon, glass, metal, or a polymer.

The polymer may be, for example, polymethylmethacrylate (PMMA), polycarbonate (PC), polystyrene (PS), polypropylene, a cyclic olefin copolymer, polynorbonene, styrene-butadiene copolymer (SBC), or acrylonitrile butadiene styrene, but is not limited thereto.

In addition, a method of forming the micro-pillars or the micro-wells in the first substrate and the second substrate is not particularly limited. For example, the first and second substrates may be formed through a photoresist process, an etching process, an injection molding process, and the like.

Referring to FIG. 2, the apparatus for analyzing a biomaterial according to the exemplary embodiment of the present invention may be operated by combining the first substrate 110 and the second substrate 120 with each other. When the first substrate 110 and the second substrate 120 are combined, the micro-pillars 111 formed in the first substrate 110 maybe inserted into the micro-wells 121 formed in the second substrate 120.

When the first substrate 110 and the second substrate 120 are combined, spacers 122 maybe disposed between the first substrate 110 and the second substrate 120 so that a space can be formed between the first substrate 110 and the second substrate 120. In the present exemplary embodiment, the spacers 122 are formed on one surface of the second substrate 120.

A biomaterial C may be attached to one surface of the micro-pillar 111. In the embodiment of the present invention, the biomaterial may refer to various biomolecules or biomaterials, and though not limited, for example, the biomolecules or biomaterials may be an arrangement of nucleic acids such as RNA, DNA, or the like, peptides, proteins, fats, organic or inorganic chemical molecules, virus particles, prokaryotic cells, cellular organelles, or the like. The cells may be, but are not limited to, for example, microorganisms, animal and plant cells, cancer cells, nerve cells, endovascular cells, immune cells, or the like.

A biomaterial C may be attached to the micro-pillar 111 while the biomaterial C is enclosed by a material capable of maintaining biological tissue and keeping a function thereof.

Various reagents maybe disposed in the micro-wells 121, and when the micro-pillars 111 are inserted into the micro-wells 121, various reagents can be supplied directly thereto. Various experiments may be performed by analyzing characteristics of the biomaterial according to the reagents.

For example, it is necessary to culture cells in order to test the efficacy of a medication candidate material for specific cells. Herein, a culture medium necessary for culturing cells may be loaded in the micro-wells 121. Then, several reagents may also be loaded in the micro-wells to supply the reagents to the cells attached to the micro-pillars, thereby testing the characteristics of the biomaterial.

In the present exemplary embodiment of the present invention, the biomaterial may be attached to the micro-pillar 111 in a three-dimensional structure. The biomaterial having the three-dimensional structure is more similar to a biological environment, thereby obtaining more accurate test results.

In addition, the present exemplary embodiment of the present invention is capable of easily washing the biomaterial after various kinds of medication treatments due to the attachment of the biomaterial to the protuded structure, as compared with a case in which the biomaterial is fixed in the micro-well. Further, the present exemplary embodiment of the present invention is capable of separating and washing the first substrate when the cells are needed to be washed, and periodically exchanging the culture medium and the reagents loaded in the micro-well.

In addition, since the plurality of micro-wells are physically shielded from one another, the culture medium or the reagents are less likely to diffuse into each other. Therefore, contamination between the micro-wells may be fundametnally prevented, thereby lowering the possibility of the occurance of errors in experimentation.

FIG. 3 is a cross-sectional view showing an apparatus for analyzing a biomaterial according to another exemplary embodiment of the present invention. The present exemplary embodiment will be described based on the above-described exemplary embodiment.

Referring to FIG. 3, a plurality of penetration holes 112 are formed in a first substrate. The plurality of penetration holes 112 may be disposed between the micro-pillars, and the number of penetration holes is not particularly limited. In addition, the penetration holes 112 may be formed at positions which do not correspond to the spacers 122. Air and vapor containing carbon dioxide necessary for the storing of the biomaterial and maintaining the functions of the biomaterial may be supplied through the penetration holes 112.

The amount of air and vapor supplied to the biomaterial may be controlled by regulating the number of penetration holes 112. In addition, when the penetration holes 112 are formed in the first substrate, the precision in the positions of the fine structures such as micro-pillars or the like can be improved in a molding process such as inection or the like.

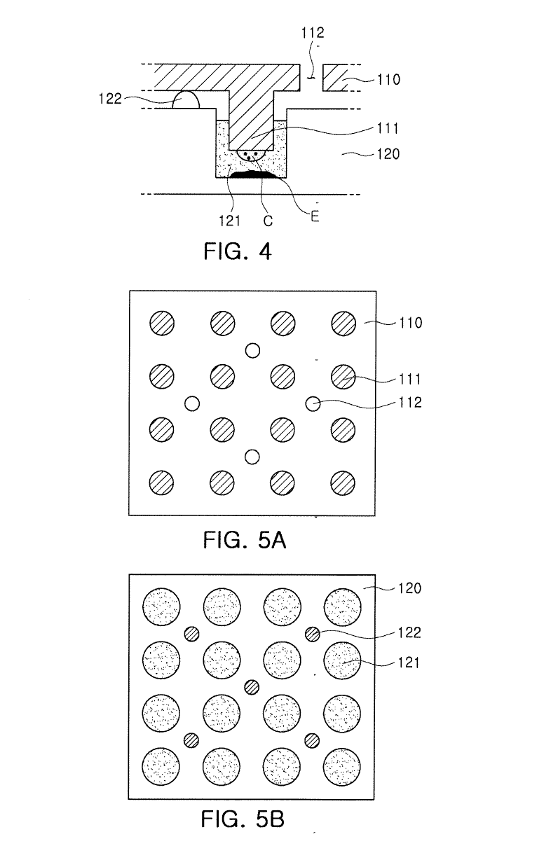

FIG. 4 is a cross-sectional view showing a portion of an apparatus for analyzing a biomaterial according to another exemplary embodiment of the present invention. This exemplary embodiment of the present invention will be described based on the above-described exemplary embodiment and other components. Referring to FIG. 4, a micro-pillar 111 is inserted into a micro-well 121 when the first substrate 110 and the second substrate 120 are combined. The biomaterial C attached to the micro-pillars 111 may be cells. Further, culture medium containing enzymes E may be loaded in the micro-wells 121.

A penetration hole 112 is formed in the first substrate 110, and a spacer 122 is formed on the second substrate 120. The penetration hole may be formed between the micro-pillars, and the spacer 112 may be formed on the periphery of the micro-well 121. The penetration hole and the spacer may be formed at positions in which they do not correspond to each other.

As described above, it is needed to supply the culture medium and various reagents in order to analyze the biomaterial.

However, when the culture medium and various reagents are stored in a culturing chamber for a long period of time during which the first substrate 110 and the second substrate 120 are combined with each other, the moisture inside the culturing chamber may be condensed between the first substrate 110 and the second substrate 120 to form water droplets. These water droplets, formed by the condensation of moisture, may move along the surfaces of the first substrate and the second substrate or permeate into the inside of the micro-well, thereby cross-contamination between the micro-wells and bubble trapping may occur.

According to the exemplary embodiments of the present invention described with reference to FIG. 1 to FIG. 4, the spacers may be disposed between the first substrate and the second substrate to form a distance between the first substrate and the second substrate.

As such, the distance is formed between the first substrate and the second substrate to form an air layer, thereby lowering the possibility of the condensation of moisture. In addition, even in the case that moisture is condensed between the first substrate and the second substrate, the condensed water droplets can be prevented from spreading across a narrow distance between the first substrate and the second substrate. Therefore, cross-contamination between the micro-wells can be prevented.

The spacers 122 may be formed on the second substrate 120, but are not limited thereto. The spacers 122 formed on the second substrate 120 may be contacted with the first substrate 110. Herein, the contact between the spacers and the first substrate may be a surface contact type or a point contact type.

The spacer shown in FIG. 4 has a curved surface, and is point-contacted with the first substrate. This can increase the area of the air layer formed between the first substrate and the second substrate, and decrease the area in which the moisture is condensed.

FIG. 5A and FIG. 5B are plan views showing a first substrate and a second substrate in an apparatus for anaylzing a biomaterial according to an exemplary embodiment of the present invention, and the positions in which the penetration holes and the spacers thereof are formed can be clearly provided by the figures.

FIG. 5A is a plan view showing the first substrate. Referring to FIG. 5A, penetration holes 112 are formed among a plurality of micro-pillars 111. The penetration holes may be, but are not limited to being, formed between two micro-pillars, of the plurality of micro-pillars, having the longest interval therebetween among the plurality of adjacent micro-pillars Also, the penegration holes may be formed between two micro-pillars formed in a diagonal direction among adjacent four micro-pillars as shown in the figure. Accordingly, the utilization of the space of the first substrate can be significantly increased.

FIG. 5B is an upper plane view showing the second substrate. Referring to FIG. 5B, spacers 122 are formed among micro-wells 121. The spacer may be, but are not limited to being, formed between two micro-wells, of the plurality of micro-wells, having the longest interval therebetween among the plurality of adjacent micro-wells, and formed such that the spacer does not correspond to the penetration hole when the first substrate and the second substrate are combined. In other words, the penetration holes and the spacers may be disposed at positions in which the penetration holes and the spacers do not contact each other when the first substrate and the second substrate are combined with each other.

In addition, the spacers may be formed on the entire surface of the first substrate or the second substrate, or formed only on the edge portion of the first substrate or the second substrate.

According to an exemplary embodiment of the present invention, the first substrate is -combined on the second substrate. Herein, when the flexibility of the first substrate is secured, the micro-pillars and the micro-wells maybe easily matched. When the spacers are formed on the first substrate, the spacers may be formed on only the edge portion of the first substrate in order to secure the flexibility of the first substrate.

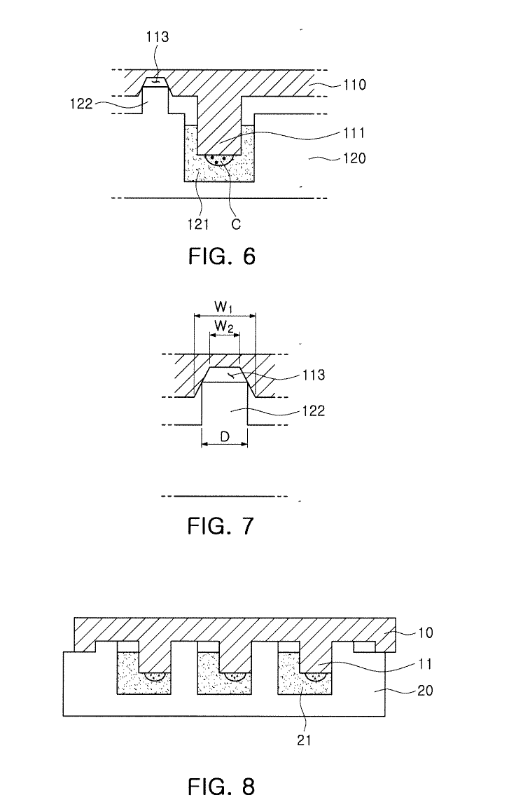

FIG. 6 is a cross-sectional view showing a portion of an apparatus for anaylzing a biomaterial according to another exemplary embodiment of the present invention, and FIG. 7 is an enlarged cross-sectional view showing structures of a spacer and a groove. This exemplary embodiment of the present invention will be described based on the above-described exemplary embodiment and other components, and a detailed description of the same or like components will be omitted.

Referring to FIG. 6 and FIG. 7, a guide groove 113 is formed in a first substrate 110 at a position corresponding to a spacer 122 formed on the second substrate 120.

Herein, the spacer 122 may be used in forming a space between the first substrate and the second substrate, and simultaneously, as a structure for the self-alignment of the micro-pillar and the micro-well. Further, some of the plurality of spacers function only as spacers for forming the space between the first substrate and the second substrate, as described above, and some of the spacers serve as structures for aligning the first substrate and the second substrate together with the guide groove.

In order that the spacer and the guide groove serve as structures for the self-alignment of the first substrate and the second substrate, only a portion of the spacers needs to be inserted into the guide groove. To achieve this, it is necessary to vary the width of any one of the spacers and the guide grooves in a depth direction or height direction thereof.

As shown in FIG. 6 and FIG. 7, the width (W) of one region of the guide groove 113 may be formed to be smaller than the width (D) of the spacer. In this exemplary embodiment, the guide groove may have a width varying in a depth direction thereof. A width (W1) of the guide groove in a surface of the first substrate is formed to be larger than the width (D) of the spacer, and a width (W2) of a bottom surface of the guide groove may be formed to be smaller than the width (D) of the spacer. As the width of the guide groove varies, there exists a region in which the guide groove is equal to the spacer in width. In order to insert only a portion of the spacer into the guide groove, a region of the guide groove having a width equal to that of the spacer, from the first substrate surface, needs to have a smaller depth than the height of the spacer.

In the early stage of the combining of the first substrate and the second substrate, the insertion of the spacer is facilitated due to a large width of the guide groove. As the combination of the first substrate and the second substrate is gradually completed, the spacer moves inwardly along the guide groove, and stops at the point of the guide groove having a width equal to that of the spacer. Therefore, a central position of the micro-pillar is naturally aligned, and the micro-pillar is inserted into the micro-well. This may be referred to as self-alignment.

FIG. 8 is a cross-sectional view showing an apparatus for anaylzing a biomaterial without spacers and guide grooves, different from the exemplary embodiment of the present invention.

In order to easily combine a first substrate 10 and a second substrate 20, a distance between the micro-pillars 11, a width of the micro-well 21, and a distance between the micro-wells need to be formed to be relatively large. The first substrate 10 and the second substrate 20 may be easily combined when the above distances are formed to be comparatively large. However, this may cause a defect in which a central axis of the micro-pillars 11 continuously move in the micro-well 21 as shown in FIG. 8, after a combination of the first substrate and the second substrate.

When the position of the micro-pillar 11 is not fixed and is moved within the micro well 21, bubbles may be generated in the reagent loaded in the micro-well. In addition, as the number of times at which the first substrate and the second substrate are combined is increased; the frequency at which bubbles are generated inside the micro-well may be rapidly increased due to the movement of the micro-pillars. In addition, cross-contamination between the micro-wells may also become worse.

On the contrary, according to an exemplary embodiment of the present invention, the first substrate and the second substrate can be easily combined and the first substrate can be fixed to the second substrate due to the self-alignment of the first substrate and the second substrate. Therefore, the embodiment of the present invention is capable of significantly reducing the movement of the micro-pillars and prevenging the generation of the bubbles inside the micro-wells and cross-contamination. In addition, because of the nature of self-alignment, a constant distance can be formed between the first substrate and the second substrate. Therefore, even though water droplets may be formed between the first substrate and the second substrate due to the condensation of moisture, the water drops may be prevented from gathering and wetting the entire surface of the first substrate or the second substrate.

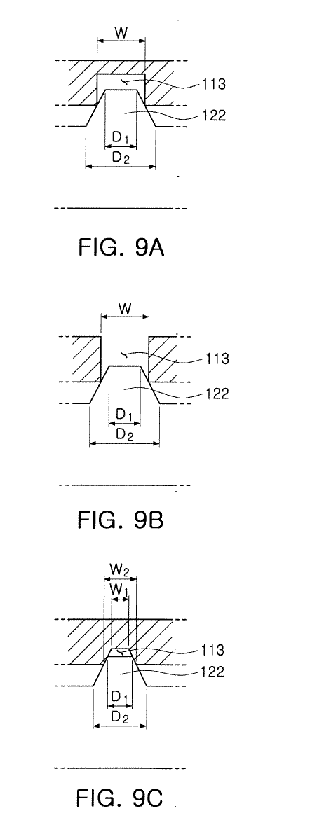

FIGS. 9A to 9C are cross-sectional views showing a spacer and a guide groove according to another exemplary embodiment of the present invention. Referring to FIGS. 9A to 9C, only a portion of the at least one spacer 122 is inserted into the guide groove 113 to have a self-alignment structure. More specially, as shown in FIG. 9A and FIG. 9B, the width of the guide groove 113 may be constant and the width of the spacer 122 may be varied in a height direction. In addition, as shown in FIG. 9B, the guide groove 113 may have a shape which passes through the first substrate.

As the width of the spacer varies from D2 to D1 in the height direction, the spacer has a region having a width equal to that of the guide groove. In addition, the height of the region of the spacer having a width equal to that of the guide groove, from the second substrate surface, needs to be smaller than the depth of the guide groove. More specially, in the early stages of the combining of the first substrate and the second substrate, the insertion of the spacer is facilitated since the width D1 of the spacer is smaller than the diameter W of the guide groove. After that, as the combination of the first substrate and the second substrate is gradually completed, the guide groove moves inwardly along the spacer and stops at the region of the spacer having a width equal to that of the guide groove. Therefore, the central position of the micro-pillar can be naturally aligned, and inserted into the micro-well.

In addition, as shown in FIG. 9B, both of the width of the guide groove 113 and the width of the spacer 122 may vary in a depth or height direction. Herein, there needs to be a region in which the guide grove is equal to the spacer in width. The depth of a region of the guide groove having a width equal to that of the spacer, from the first substrate surface, needs to be smaller than the height of the spacer.

In the early stages of the combining of the first substrate and the second substrate, the insertion of the spacer is facilitated since the width D1 of the spacer is smaller than the diameter W1 of the guide groove. As the combination of the first substrate and the second substrate is gradually completed, the guide groove moves along the spacer, and stops at the region of the spacer having a width equal to that of the guide groove. Therefore, a central position of the micro-pillar can be naturally aligned, and the micro-pillar can be inserted into the micro-well.

The shapes of the spacer and the guide groove may not be limited thereto, and may be changed in various structures allowing for self-alignment.

As set forth above, the exemplary embodiments of the present invention are capable of attaching the biomaterial to the micor-pillar in a three-dimentional structure, and thus, performing tests under conditions similar to those of a biological environment.

In addition, the present exemplary embodiments of the present invention allow for the easily washing of biomaterials after various kinds of medication treatments due to the attachment of the biomaterial to the micro-pillar having the protuded structure. Further, the present exemplary embodiments of the present invention are capable of separating and washing the first substrate, when the biomateiral needs to be washed. Also, the present exemplary embodiments of the present invention are capable of periodically exchanging the culture medium and the reagents provided in the micro-well.

Further, since the plurality of micro-wells are physically shielded from one another, the culture medium or the reagents are less likely to diffuse into each other. Therefore, contamination between the micro-wells may be fundamentally prevented, thereby lowering the possibility of errors occurring during experimentation.

Further, the distance between the first substrate and the second substrate can be formed by disposing the spacers between the first substrate and the second substrate. When the distance is formed between the first substrate and the second substrate, an additive air layer may be formed, thereby lowering the possibility of the condensation of moisture. Also, even though the moisture is condensed between the first substrate and the second substrate, the condensed water drop can be prevented from spreading across a narrow distance between the first substrate and the second substrate. Therefore, cross-contamination between the micro-wells can be prevented.

In addition, when penetration holes are formed in the first substrate, air and vapor containing carbon dioxide can be supplied to the biomaterial through the penetration holes.

Further, when the guide groove is formed in the first substrate, the first substrate and the second substrate can be easily combined with each other and the first substrate can be fixed to the second substrate due to self-alignment. Therefore, the exemplary embodiments of the present invention are capable of significantly reducing the movement of the micro-pillars and preventing the generation of bubbles inside the micro-well and cross-contamination. Also, because of the nature of self-alignment, a constant distance can be formed between the first substrate and the second substrate. Therefore, even in the case that water drops are formed between the first substrate and the second substrate due to the condensation of moisture, water drops may be prevented from gathering and wetting the entire surface of the first substrate or the second substrate may be avoided.

While the present invention has been shown and described in connection with the exemplary embodiments, it will be apparent to those skilled in the art that modification and variation can be made without departing from the spirit and scope of the invention as defined by the appended claims.

What is claimed:

1. An apparatus for analyzing a biomaterial, the apparatus comprising:
a first substrate comprising a plurality of micro-pillars formed on a surface of the substrate, wherein said micropillars protrude from said surface to a predetermined height, and wherein a biomaterial is attached to a surface of the micro-pillar;
a second substrate comprising a plurality of micro-wells, the micro-pillars being insertable into the micro-wells when the first substrate and the second substrate are combined with each other; and
at least one spacer disposed between the first substrate and the second substrate when the first substrate and the second substrate are combined with each other.

2. The apparatus of claim 1, wherein the one spacer is formed on the surface of the second substrate.

3. The apparatus of claim 2, wherein the spacer is surface-contacted or point-contacted with the first substrate.

4. The apparatus of any one of claims 1 to 3, wherein the first substrate further comprises a penetration hole formed between the micro-pillars thereof.

5. The apparatus of any one of claims 1 to 4, wherein the spacer is formed at one of the ends of the first substrate or the second substrate.

6. The apparatus of claim 1, wherein the spacer is formed on the second substrate, and the first substrate further comprises a penetration hole formed between the micro-pillars thereof.

7. The apparatus of claim 1, wherein the spacer is formed on the second substrate, and wherein the first substrate further comprises a guide groove into which a portion of the spacer is inserted, the guide groove being formed at a position of the first substrate corresponding to the spacer.

8. The apparatus of claim 7, wherein the guide groove has a width that varies over the depth of groove wherein the width of one region of the guide groove is equal to the width of the spacer such that only the portion of the at least one spacer is inserted into the guide groove.

9. The apparatus of claim 7, wherein the spacer has a width that varies over the height wherein the width of one region of the spacer is equal to the width of the guide groove such that only the portion of the at least one spacer is inserted into the guide groove.

10. The apparatus of claim 7, wherein the guide groove has a depth smaller than the height of the at least one spacer.

11. The apparatus of claim 7, wherein the guide groove is formed to penetrate the first substrate.

12. The apparatus of claim 7, wherein the width of the guide groove and the width of the spacer vary in a height or depth direction thereof, the width of one region of the guide groove being equal to the width of one region of the at least one spacer such that only the portion of the at least one spacer is inserted into the guide groove.

13. The apparatus of any one of claims 1 to 12, wherein the biomaterial is a cell.

14. The apparatus of any one of claims 1 to 13, wherein the biomaterial is encapsulated in a three-dimensional matrix material.

15. The apparatus of any one of claims 1 to 14, wherein the microwell comprises a reagent capable of interacting with the biomaterial.

16. The apparatus of claim 15, wherein the reagent is selected from the group consisting of an enzyme, a small molecule, a biopolymer or a combination thereof.

17. The apparatus of claim 16, wherein the enzyme is a cytochrome P450 enzyme.

18. A method for analyzing a biomaterial, the method comprising:
combining a first substrate comprising a biomaterial attached to a surface thereof with a second substrate comprising a reagent in micro-wells such that the biomaterial is brought into contact with the reagent, wherein at least one spacer is disposed between the first substrate and the second substrate when the first substrate and the second substrate are combined with each other;
wherein the first substrate comprises a plurality of micro-pillars formed on a surface of the substrate, wherein said micropillars protrude from said surface to a predetermined height, and wherein a biomaterial is attached to a surface of the micro-pillar;
and wherein the second substrate comprises a plurality of micro-wells, the micro-pillars being insertable into the micro-wells when the first substrate and the second substrate are combined with each other; and
wherein said method further comprising detecting a reaction between the biomaterial and the reagent.

19. The method of claim 18, wherein the first substrate further comprises a penetration hole formed between the micro-pillars thereof.

20. The method of any one of claims 18 to 19, wherein the spacer is formed at one of the ends of the first substrate or the second substrate.

21. The method of claims 18 to 20, wherein the first substrate further comprises a guide groove into which a portion of the spacer is inserted.

22. The method of any one of claims 18 to 21, wherein the biomaterial is encapsulated in a three-dimensional matrix material.

23. The method of any one of claims 18 to 22, wherein the wherein the reagent is selected from the group consisting of an enzyme, a small molecule, a biopolymer or a combination thereof

24. The method of claim 23, wherein the enzyme is a cytochrome P450 enzyme.
